(19) United States
(12) Patent Application Publication
(10) Pub. No.: US 2016/0089067 A1
(43) Pub. Date: Mar. 31, 2016

(54) NEAR INFRARED OXYGEN CONCENTRATION SENSOR FOR PALPATION

(71) Applicant: NATIONAL UNIVERSITY CORPORATION HAMAMATSU UNIVERSITY SCHOOL OF MEDICINE, Shizuoka (JP)

(72) Inventor: Naohiro Kanayama, (US)

(21) Appl. No.: 14/892,683

(22) PCT Filed: May 13, 2014
(86) PCT No.: PCT/JP2014/062677

§ 371 Date: Nov. 20, 2015

(30) Foreign Application Priority Data
May 24, 2013 (JP) 2013-109604

(51) Int. Cl. A61B 5/1455; A61B 5/00; A61B 5/1464; A61B 5/145

(52) U.S. Cl. CPC A61B 5/14552; A61B 5/14546; A61B 5/6826; A61B 5/1464

(57) ABSTRACT

A near-infrared oxygen concentration sensor for palpation 1 to be attached to a finger pad on a leading end side from a distal interphalangeal joint of a use's finger includes: a base material 2 to be attached to a finger pad; a light emitting unit 4 that is disposed on the base material and that emits light having at least two wavelengths, including near-infrared light, to a test subject; light receiving units 5a and 5b that are disposed on the base material and that receives a measurement light from the light emitting element through the test subject; and a light receiving unit 3 that is disposed at least between the light emitting unit or the light receiving unit and the finger pad and that prevents the measurement light having passed through the user's finger from being led to the light receiving unit.

### TECHNICAL FIELD

The present invention relates to a near-infrared oxygen concentration sensor that measures, with near-infrared light, at least any one of an oxygenated hemoglobin concentration, a deoxygenated hemoglobin concentration, and an oxygen saturation in the human body. In particular, the present invention relates to a near-infrared oxygen concentration sensor for palpation having a structure suitable to be used during palpation.

### BACKGROUND ART

Stresses during labor or by labor pains cause fetuses to suffer from hypoxemia leading to fetal dysfunction in some cases. In very severe cases, fetuses may sometimes have neonatal cerebral hypoxia leading to cerebral palsy. Therefore, monitoring the oxygen kinetics of fetuses is the best method for understanding the state of fetuses. A technology known in the art for non-invasively measuring an oxygen saturation includes near-infrared spectroscopy. A method of transvaginally observing the oxygen kinetics of fetuses using this near-infrared radiation has been attempted in the past. Specifically, there is known a method of allowing, after amniorrhexis, a sensor having a length of 4 cm with a light transmitter and a light receiver to pass along a cervical canal, and to be attached to a head via a forehead of a fetus (Patent Literature 1).

However, insertion of the sensor into a uterine involves various problems such as: a risk of infections or the like; frequent occurrence of failing to be successfully adhered to the forehead of a fetus; and failure in measurement due to the sensor shifting in position caused by a fetus descending as labor proceeds. Therefore, this has not been used for clinical applications. A method is sought in which attaching to a fetus skin can be simply and reliably achieved, and measurement can be performed irrespective of the descending of a fetus. A method is also sought for simply and reliably measuring the oxygen concentrations of sites in body cavities (such as in oral cavities and rectums) and sites (such as hearts) under surgery, other than the oxygen concentration of a fetus in a uterine.

In a near-infrared oxygen concentration sensor, reliable contact between the sensor and the surface of a test subject is extraordinarily important. A number of extracorporeal measurement techniques are known (Patent Literatures 2 and 3). However, it is difficult to use these techniques as they are for measuring sites in body cavities. Also, as a diagnostic apparatus used in palpation, an ultrasonic diagnostic apparatus for palpation is known (Patent Literature 4). However, since a sensor itself is large in size, it is difficult to use the ultrasonic diagnostic apparatus for palpation without damaging the operability of palpation. Furthermore, oxygen concentrations cannot be measured with ultrasonic waves.

Patent Literature 5 discloses a technique of attaching an optical sensor to a finger for obtaining a plethysmogram. However, Patent Literature 5 is not for measuring the oxygen concentrations of palpation sites. For this reason, there is no description on, for example, a light source having a plurality of wavelengths, and shielding the oxygen concentration information on a user's finger side. Furthermore, Patent Literature 5 discloses a sphygmomanometer, and therefore requires a pressure sensor.

### CITATION LIST

### Patent Literature

PATENT LITERATURE 1: JP-A-04-226639

PATENT LITERATURE 2: WO 2007/139192 A

PATENT LITERATURE 3: WO 2012/115210 A

PATENT LITERATURE 4: JP-A-02-307437

PATENT LITERATURE 5: JP-A-2006-239114

### SUMMARY OF INVENTION

### Problems to be Solved by the Invention

In the pulse oximeter for fetuses disclosed in Patent Literature 1, a sensor is inserted from the outside into a uterine. For this reason, it is difficult to reliably bring the sensor into contact with the skin of a fetus, and there is also a risk of infections or the like. The near-infrared oxygen concentration sensors disclosed in Patent Literatures 2 and 3 are configured to perform extracorporeal measurement, and therefore cannot be used as they are for measuring the oxygen concentrations of the sites in body cavities. The ultrasonic probe for palpation disclosed in Patent Literature 4 is configured to perform diagnosis with ultrasonic waves, and therefore cannot be used for measuring the oxygen concentrations of the sites in body cavities. Also, in the structure of Patent Literature 4, a ultrasonic probe being relatively larger in size than a fingertip is attached to a fingertip. For this reason, operability of palpation may be damaged.

The present invention has been made for solving the above-described problems. An object of the present invention is to provide an oxygen concentration sensor for palpation as described below. This oxygen concentration sensor for palpation reliably measures an oxygen concentration (an oxygenated hemoglobin concentration, a deoxygenated hemoglobin concentration, and an oxygen saturation, and the like) of a measurement target site while minimizing an influence on the operability of palpation and reliably bringing the sensor into contact with the measurement target site.

### Solutions to the Problems

In order to solve the above described problems, the present invention includes the structure as below.

A near-infrared oxygen concentration sensor for palpation that is to be attached to a finger pad on a leading end side from a distal interphalangeal joint of a user's finger and that measures an oxygen concentration of a palpation target site during palpation, includes: a base material to be attached to the finger pad; a light emitting unit that is disposed on the base material and that emits light having at least two wavelengths, including near-infrared light, to a test subject; a light receiving unit that is disposed on the base material and that receives measurement light from the light emitting element through the test subject; and a light shielding unit that is disposed at least between the light emitting unit or the light receiving unit and the finger pad.

As the finger, an index finger or a middle finger may be suitably used. However, the finger is not limited to these.

The finger pad is a portion having fingerprints on a surface that is on a leading end side from a distal interphalangeal joint of the finger and on an opposite side to a nail.

As the base material, a flat plate-like base plate may be suitably used. However, the base material is not limited to this.

As the light emitting unit, an LED may be suitably used. However, the light emitting unit is not limited to this. An optical fiber may be alternatively used so that light is externally led.

As the wavelength of the light emitted from the light emitting unit, 735 nm and 870 nm are suitably used. However, this wavelength is not limited to this, as long as it enables measurement of an oxygen concentration in a body tissue.

As the light receiving unit, photodiode or phototransistor may be suitably used. However, the light receiving unit is not limited to this. The light receiving element may be distantly disposed via an optical fiber or the like.

The light shielding unit prevents measurement light having passed through a user's finger from being led to the light receiving unit.

A material of the light shielding unit is not particularly limited, as long as it can prevent the measurement light from a user's finger from being received. An example thereof may include a black rubber material. The light shielding unit may be disposed separately from the base material, or the base material itself may have light shielding properties.

The number of light emitting units may be one, or may be two or more. The number of light receiving units may also be one, or two or more.

The minimum distance between the light emitting unit and the light receiving unit is preferably 3 mm or more, and the maximum distance therebetween is preferably 15 mm or less.

When the light emitting unit or the light receiving unit is plurally present, the minimum distance is a distance for a combination of the light emitting unit and the light receiving unit having the smallest distance. When the light emitting unit or the light receiving unit has a predetermined area, the minimum distance is a minimum distance between the ends of the light emitting unit and the light receiving unit.

When the light emitting unit or the light receiving unit is plurally present, the maximum distance is a distance for a combination of the light emitting unit and the light receiving unit having the largest distance. When the light emitting unit or the light receiving unit has a predetermined area, the maximum distance is a maximum distance between the ends of the light emitting unit and the light receiving unit.

A surface with which a test subject is brought into contact in the near-infrared oxygen concentration sensor for palpation may be flat, or may have a concavo-convex structure where the light emitting unit or the light receiving unit projects. When the surface of the sensor has the concavo-convex structure, the light emitting unit and the light receiving unit can be brought into contact with the surface of a test subject by pushing its way through body hair (such as hair on a head of a fetus) during palpation. Therefore, operability is enhanced. On the other hand, the surface of the sensor may be sometimes preferably flat depending on the measurement target site. An optimum surface structure may be selected depending on the application.

The present invention has a peculiar structure for being attached to a finger pad on a leading end side from a distal interphalangeal joint of a user's finger. In the present invention, a sensor part can be brought into contact with a measurement target site in a body cavity to measure an oxygen concentration in a tissue of the measurement target site, without damaging the operability of palpation. Specifically, the light shielding unit is disposed on the back side (the user's finger side) of the sensor part (the light emitting unit and the light receiving unit), thereby enabling information from the user's tissue to be shielded so that only information from the test subject can be acquired. The maximum distance between the light emitting unit and the light receiving unit is preferably 15 mm or less for enabling the attachment to a finger pad. The distance between the light emitting unit and the light receiving unit needs to be a certain distance or more for acquiring the information in a tissue. In a commercially available pulse oximeter for fetuses (NEELCOR Incorporated, Oxifirst) corresponding to Patent Literature 1, a sensor part has a length of approximately 40 mm. On the other hand, in the present invention, an algorithm for calculating an oxygen concentration is elaborated to realize a maximum distance of 15 mm or less. Furthermore, since the present invention is for palpation, a user generally wears a transparent or translucent diagnostic glove which transmits near-infrared radiation when used. In the present invention, light from the light emitting unit is prevented from passing through a glove and being directly led to the light receiving unit by: defining the minimum distance between the light emitting unit and the light receiving unit to be 3 mm or more, and using a glove which transmits near-infrared radiation.

The present invention can be suitably used for measuring the oxygen concentration of a fetus by being brought into contact with a scalp of the fetus in a uterine. A medical doctor frequently performs a pelvic examination for seeing the progress of labor. When performing a pelvic examination, a medical doctor touches a scalp of a fetus for diagnosis. At this time, the near-infrared oxygen concentration sensor for palpation according to the present invention is attached to a fingertip so that the sensor can be reliably brought into contact with the scalp of a fetus, thereby enabling the oxygen concentration of the fetus to be measured. The sensor according to the present invention is extraordinarily small in size. For this reason, the diagnosis and the measurement of an oxygen concentration can be performed by the same procedure as that in diagnosis by a regular pelvic examination.

It is noted that the present invention can be used for, other than a fetus in a uterine, any site that a medical doctor can touch. Examples of such a site may include sites in body cavities (in oral cavities, rectums, and the like) and sites under surgery (for example, hearts). When used during a myocardial infarction surgery, it can be understood which portion of the heart has a decreased oxygen concentration. Another example may include all intraperitoneal or intrathoracic organs that a medical doctor can tough during surgery. Examples of such organs may include a liver, stomach, spleen, pancreas, and intestine. Furthermore, when used in oral cavities or axillae, the oxygen concentration in a portion closer to a brain can be directly measured. Especially, the condition of a severe patient, which has been difficult to measure using a known pulse oximeter to be attached to a finger, can be quickly diagnosed. Furthermore, a medical doctor can identify a site while touching, and measure the oxygen concentration of the site. For this reason, the present invention can be used on a skin in any site on the body surface. For example, the present invention can also be used to understand oxygen kinetics in each site of the skin of a newborn baby.

The present invention has the following preferred embodiment.

The present invention has an operation unit that calculates at least any one of an oxygenated hemoglobin concentration, a deoxygenated hemoglobin concentration, and an oxygen saturation of a test subject, based on measurement light from the light receiving unit.

The present invention also has the following preferred embodiment.

The number of the light emitting units and the light receiving units in total is three or more.

The operation unit calculates at least any one of an oxygenated hemoglobin concentration, a deoxygenated hemoglobin concentration, and an oxygen saturation of a test subject, based on measurement light in a plurality of distances between the light emitting units and the light receiving units.

The number of the light emitting units and the number of the light receiving units are each one or more. Therefore, a combination of the light emitting unit and the light receiving unit in which the number of the light emitting units and the light receiving units in total is three or more may include: one light emitting unit and a plurality of light receiving units; a plurality of light emitting units and one light receiving unit; or a plurality of light emitting units and a plurality of light receiving units. A combination of the light emitting unit and the light receiving unit is preferably one light emitting unit and a plurality of light receiving units, and further preferably one light emitting unit and two light receiving units. When the number of light emitting units is two or more, variations in an element of the light emitting unit itself are likely to have an influence. For this reason, it is preferred that the number of light emitting units is one, and the number of light receiving units is two or more.

The present invention can also be used as a pulse oximeter. More suitably, there may be used an operation of obtaining spatial slope S based on measurement light in a plurality of distances between the light emitting units and the light receiving units as disclosed in Patent Literatures 2 and 3, and then obtaining an absorbance in a tissue. A pulse oximeter can measure only the oxygen concentration of a portion having a large pulsation (for example, arterials). On the other hand, with the operation using spatial slope S, the oxygen concentration for even a portion having a small pulsation can be measured. For this reason, the oxygen concentration of a body surface tissue or the like can be measured with more certainty. This is particularly effective when measuring the oxygen concentration of a fetus having a risk of hypoxemia. Furthermore, with the operation using spatial slope S, there can be measured absolute values of not only the oxygen saturation but also the oxygenated hemoglobin concentration and the deoxygenated hemoglobin concentration, thereby enabling more diagnostic information to be obtained.

The present invention has the following preferred embodiment.

The minimum distance between the light emitting unit and the light receiving unit is 3 mm or more, and the maximum distance therebetween is 15 mm or less.

The present invention has the following preferred embodiment.

The present invention has a fixing unit that fixes the base material to the finger pad.

The fixing unit is not particularly limited, as long as it can relatively fix the base material to the finger pad. Suitable examples may include fixing with an adhesive tape, fixing with a band, and fixing the base material to a finger cot configured to fit around a finger. The fixing unit may also function as the light shielding unit.

The present invention has the following preferred embodiment.

At least a portion containing the base material, the light emitting unit and the light receiving unit is disposable.

The sensor according to the present invention may be inserted into a body cavity of a test subject. For this reason, when at least a portion to be inserted into the body cavity is disposable, a risk of infections can be prevented.

The present invention has the following preferred embodiment.

The near-infrared concentration sensor for palpation is used while wearing a glove which transmits near-infrared light.

The operation unit has a unit that cancels an influence by the glove on the measurement light.

The glove which transmits near-infrared light may be, for example, transparent or white color, and made of plastic or vinyl.

When a user wears a glove, the glove comes to lie between the sensor part and the measurement target site. In a pulse oximeter, variations due to pulsations are calculated, and therefore an influence by the glove can be canceled. In the operation using spatial slope S, a sufficient distance (3 mm or more) between the light emitting unit and the light receiving unit allows the amount of light absorbed by a glove to be independent from the distance. For this reason, the influence by the glove can be removed from the measurement light in a plurality of distances between the light emitting units and the light receiving units.

The present invention has the following preferred embodiment.

As a signal cable to be connected to the light emitting unit and the light receiving unit, a flat cable is used.

### Advantageous Effects of the Invention

According to the above-described configuration of the present invention, the oxygen concentration sensor for palpation can measure an oxygen concentration (an oxygenated hemoglobin concentration, a deoxygenated hemoglobin concentration, an oxygen saturation, and the like) of a measurement target site, while minimizing an influence on the operability of palpation and reliably bringing the sensor into contact with the site.

### BRIEF DESCRIPTION OF THE DRAWINGS

FIG. 1 is a front view of an embodiment of the present invention.

FIG. 2 is a side view of an embodiment of the present invention.

FIG. 3 is an appearance view of an embodiment of the present invention.

FIG. 4 is a system diagram of an embodiment of the present invention.

FIG. 5 is an illustrative view of measurement light propagation paths in an embodiment of the present invention.

### DESCRIPTION OF EMBODIMENTS

Hereinafter, suitable embodiments of the near-infrared oxygen concentration sensor for palpation according to the present invention will be described with reference to the drawings.

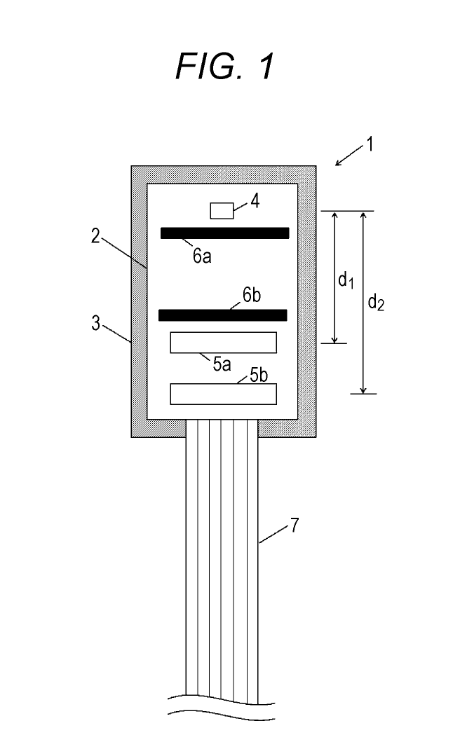

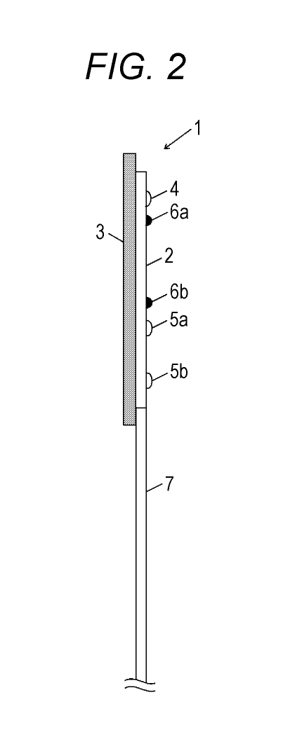

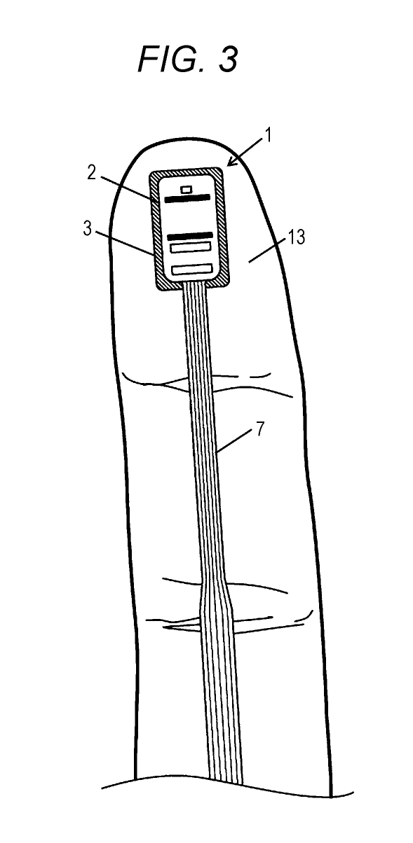

FIG. 1 is a front view of a near-infrared oxygen concentration sensor for palpation according to the present embodiment. FIG. 2 is a side view of a sensor body 1. FIG. 3 is an appearance view of the near-infrared oxygen concentration sensor for palpation according to the present embodiment which is attached to a finger.

As illustrated in FIG. 3, the sensor body 1 has a shape and size that fit in a finger pad 13 (on a leading end side from a distal interphalangeal joint) of a user (such as a medical doctor). The sensor body 1 is fixed to the finger pad 13 of a user. A flexible flat cable 7 is led from the sensor body 1. The flat cable 7 is connected to a connector (not shown) on a palm side from the base of a finger. The flat cable can also extend along a knuckle. Since the sensor body 1 has a shape and size that fit in the finger pad 13 of a user, the user can measure the oxygen concentration of a touched site without damaging the sense and operability by palpation. In actual use, a user wears a glove used for medical examinations or the like. The glove can transmit near-infrared radiation. The glove to be used is transparent, translucent, or white.

As illustrated in FIGS. 1 and 2, the sensor body 1 includes a base plate 2, a light shielding body 3 disposed on a back surface of the base plate 2, a light emitting element 4 disposed on the base plate 2, a first light receiving element 5a, a second light receiving element 5b, a first light shielding wall 6a, and a second light shielding wall 6b. The first light receiving element 5a is disposed on the base plate 2, and spaced apart from the light emitting element 4 by a predetermined distance. The second light receiving element 5b is disposed on the base plate 2, and spaced apart from the light emitting element 4 further than the first light receiving element 5a. The first light shielding wall 6a and the second light shielding wall 6b are disposed between the light emitting element 4 and the first light receiving element 5a. The flat cable 7 is connected to the base plate 2 of the sensor body 1. The flat cable 7 is connected to a sensor controller 8 described later.

Examples of a material of the base plate 2 may include epoxy or polyimide. For enhancing the contact properties to the surface of a test subject, the base plate is preferably flexible. However, when the sensor body 1 is sufficiently small, the base plate may be hard. The base plate may have a size that fits in the finger pad of a user. In the present embodiment, the base plate has a length of approximately 10 mm and a width of approximately 5 mm.

The light shielding body 3 prevents oxygen concentration information of a user's finger from arriving at the sensor body 1. The sensor body 1 is thin, and is to be attached to a user's finger. For this reason, light from the light emitting element 4 can be emitted to the user's finger. When the light emitted to the user's finger is received by the light receiving element 5, the oxygen concentration information of the user is also included. Therefore, disposition of the light shielding body 3 between the sensor body 1 and the user's finger pad shields the light information from the user's finger. Since the measurement light from the user's finger only needs to be prevented from arriving at the light receiving element 5, various arrangements are conceivable such as shielding only the back surface of the light emitting element 4, shielding only the back surface of the light receiving element 5, or shielding the whole back surface of the base plate 2. Also, the base plate 2 may include a light shielding material so that the base plate 2 itself also functions as a light shielding body. In the present embodiment, a black rubber material is used as a material of the light shielding body 3. However, the material of the light shielding body 3 is not limited to this, as long as it has light shielding properties.

In the present embodiment, an LED that emits light having wavelengths of 735 nm and 870 nm is used as the light emitting element 4. The light emitting element 4 is not particularly limited, as long as it is a light source capable of emitting light having at least two wavelengths into a test subject.

In the present embodiment, photodiode is used as the light receiving element 5. The distance (first distance d₁) between the light emitting element 4 and the first light receiving element 5a is approximately 6 mm. The distance (second distance d₂) between the light emitting element 4 and the second light receiving element 5b is approximately 8 mm. The light receiving element 5 is not particularly limited, as long as it can receive light from the inside of a test subject.

The light shielding wall 6 is disposed between the light emitting element 4 and the light receiving element 5. The light shielding wall 6 prevents direct light from the light emitting element 4 from being detected by the light receiving element 5. In the present embodiment, the first light shielding wall 6a is arranged on a side closer to the light emitting element 4, and the second light shielding wall 6b is arranged on a side closer to the first light receiving element 5a.

The flat cable 7 is used for, for example, connection of an electronic circuit. An example of the flat cable 7 to be used may include polyimide. The flat cable 7 may be connected to the base plate 2 via a connector or the like, or may be unified with the base plate 2. The end of the flat cable 7 opposite to the sensor body 1 is to be connected to a connector (not shown) lying on a metacarpus beyond the base of a finger. The position of the connector is not particularly limited, as long as it does not hinder palpation, and may be set in an arm part beyond a palm. The flat cable 7 is connected to, beyond the connector, a sensor controller 8 described later. In the present embodiment, the flat cable 7 has a width of approximately 3 mm.

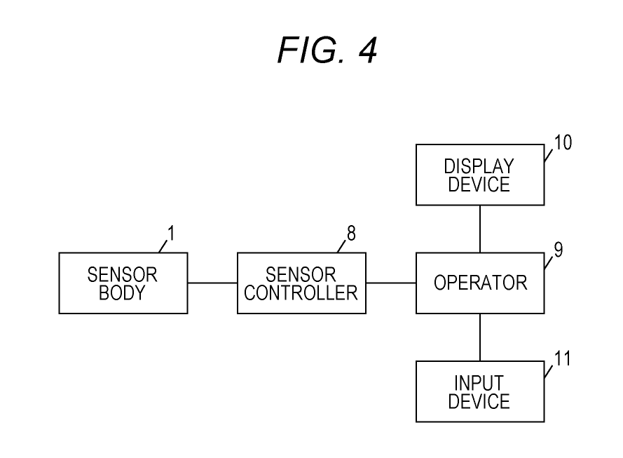

A system configuration of the present embodiment will be described with reference to FIG. 4. A near-infrared oxygen concentration measurement system according to the present embodiment includes a sensor controller 8, an operator 9, a display device 10, and an input device 11. The sensor controller 8 is connected to the sensor body 1 for controlling the sensor body 1. The operator 9 is connected to the sensor controller 8 for analyzing signals from the sensor controller 8 and calculating an oxygen concentration and the like. The display device 10 displays the oxygen concentration and the like calculated by the operator 9. The input device 11 inputs a parameter and the like to the operator 9.

The sensor controller 8 has, for example, a driver for driving the light emitting element 4 and an amplifier for amplifying signals from the light receiving element 5. The timing of light emitting by the light emitting element 4 and the timing of light receiving by the light receiving element 5 may be controlled by the sensor controller 8, or may be controlled by the operator 9. Analog signals from the light receiving element 5 may be digitized in either the sensor controller 8 or the operator 9.

As the operator 9, a PC (personal computer) or the like is used. The operator 9 may be unified with the sensor controller 8 to form a specialized machine. In the operator 9, a pulse oximeter method may be used, or a spatially resolved method may be used. In the pulse oximeter method, an oxygen saturation and the like are obtained from variations in absorbance due to pulsation. In the spatially resolved method, an oxygenated hemoglobin concentration, a deoxygenated hemoglobin concentration, an oxygen saturation, and the like are obtained by taking advantage of a spatial slope described later. The display device 10 is not particularly limited, as long as it can display operation results. As the display device 10, an LCD or the like is used. The input device 11 is also not particularly limited, as long as it is a device capable of inputting. As the input device 11, a keyboard, a mouse, a touch panel, or the like is used.

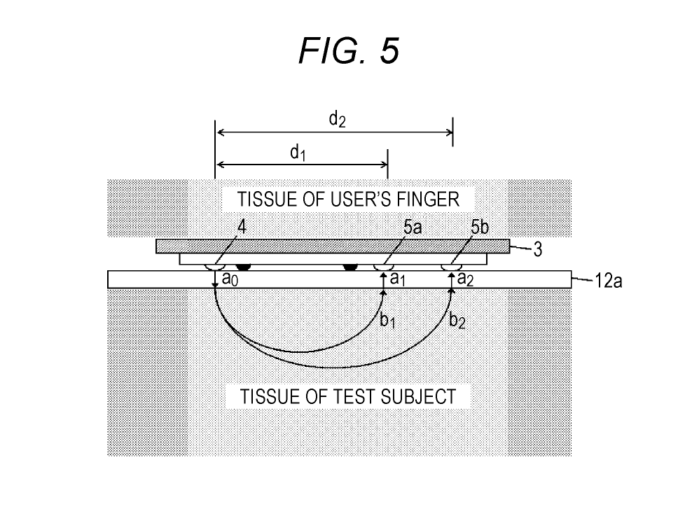

The spatially resolved method, which is an oxygen concentration calculating method suitably used in the present embodiment, will be described with reference to FIG. 5. An operation of this algorithm is executed in the operator 9.

Light emitted from the light emitting element 4 passes through light path a₀ in the glove 12, and irradiates a tissue of a test subject. The light emitted onto the tissue of a test subject is absorbed and scattered in the tissue, and passes through light path a₁ and light path a₂ in the glove 12 via light path b₁ and light path b₂. Thereafter, the light is received by the first light receiving element 5a and the second light receiving element 5b. In the drawing, the light path b₁ and light path b₂ are linearly indicated for convenience. However, light is actually propagated while being scattered in the tissue. For this reason, the light paths are complicated. The light shielding body 3 is disposed on the user's finger side. According to the light shielding body 3, the light emitted from the light emitting element 4 is prevented from being propagated in a tissue of the user's finger and led to the light receiving element 5.

Regarding the spatially resolved method, one of the inventors of this application found that the absorption coefficient of the light in a human tissue can be expressed by a function of spatial slope S, based on the diffusion theory, various simulations, and the like. Details thereof are described in Patent Literatures 2 and 3.

When light receiving intensity in the first light receiving element 5a is I₁, light receiving intensity in the second light receiving element 5b is I₂, a distance between the light emitting element 4 and the first light receiving element 5a is d₁, and a distance between the light emitting element 4 and the second light receiving element 5b is d₂, spatial slope S is defined by

S=ln(I₁/I₂)/(d₂−d₁) (1)

Since d₁ and d₂ are known, obtaining only a ratio between I₁ and I₂ by measurement enables spatial slope S to be obtained. Once spatial slope S is obtained, the absorption coefficient of the light in an tissue is obtained by using a look-up table or the like. For this reason, once an absorption coefficient for each wavelength is obtained, the oxygenated hemoglobin concentration and the deoxygenated hemoglobin concentration can be calculated, and the oxygen saturation, which is a ratio between the oxygenated hemoglobin concentration and the deoxygenated hemoglobin concentration, can also be calculated. It is noted that since the distance between the light emitting element 4 and the light receiving element 5 in the present embodiment (for example, 15 mm or less) is shorter than that known in the art, an improved algorithm utilizing the transport theory may be employed.

The near-infrared oxygen concentration sensor for palpation according to the present embodiment is used while wearing the glove 12 in an actual use form. The glove 12 extends, as illustrated in FIG. 5, between the light emitting element 4 and the light receiving element 5, and has an influence on light receiving signals. However, when the distance between the light emitting element 4 and the light receiving element 5 is sufficient (for example, 3 mm or more), light path a₀, light path a₁, and light path a₂ are each vertical to the light emitting surface and the light receiving surface, and considered to have an identical length. These pieces of information can be used to cancel an influence by the glove 12.

An embodiment of the present invention has been described above. However, the present invention is not limited to this. Certainly, various modifications and changes can be made within the scope of the technical ideas as described in the claims.

### DESCRIPTION OF REFERENCE SIGNS

1: sensor body, 2: base plate, 3: light shielding body, 4: light emitting element, 5a: first light receiving element, 5b: second light receiving element, 6a: first light shielding wall, 6b: second light shielding wall, 7: flat cable, 8: sensor controller, 9: operator, 10: display device, 11: input device, 12: glove, 13: finger pad

1. A near-infrared oxygen concentration sensor for palpation that is to be attached to a finger pad on a leading end side from a distal interphalangeal joint of a user's finger and that measures an oxygen concentration of a palpation target site while the palpation is being performed, comprising:
a base material to be attached to the finger pad;
a light emitting unit that is disposed on the base material and that emits light having at least two wavelengths, including near-infrared light, to a test subject;
a light receiving unit that is disposed on the base material and that receives measurement light from the light emitting unit through the test subject;
a light shielding unit that is disposed at least between the light emitting unit or the light receiving unit and the finger pad and that prevents the measurement light passing through the user's finger from being led to the light receiving unit;
a fixing unit that fixes the base material to the finger pad; and
an operation unit that calculates at least any one of an oxygenated hemoglobin concentration, a deoxygenated hemoglobin concentration, and an oxygen saturation of the test subject, based on the measurement light from the light receiving unit, wherein
the number of the light emitting units and the light receiving units in total is three or more, and
the operation unit calculates at least any one of an oxygenated hemoglobin concentration, a deoxygenated hemoglobin concentration, and an oxygen saturation of the test subject, based on the measurement light in a plurality of distances between the light emitting units and the light receiving units.

2. (canceled)

3. (canceled)

4. The near-infrared oxygen concentration sensor for palpation according to claim 1, wherein
a minimum distance between the light emitting unit and the light receiving unit is 3 mm or more, and a maximum distance between the light emitting unit and the light receiving unit is 15 mm or less.

5. (canceled)

6. The near-infrared oxygen concentration sensor for palpation according to claim 1, wherein
at least a portion containing the base material, the light emitting unit, and the light receiving unit is disposable.

7. The near-infrared oxygen concentration sensor for palpation according to claim 1, wherein
the near-infrared concentration sensor for palpation is used while wearing a glove which transmits near-infrared light, and
the operation unit has a unit that cancels an influence by the glove on the measurement light.

8. The near-infrared oxygen concentration sensor for palpation according to claim 1, wherein
a flat cable is used as a signal cable to be connected to the light emitting unit and the light receiving unit.

9. The near-infrared oxygen concentration sensor for palpation according to claim 1, wherein
the number of light emitting units is one, and the number of light receiving units is two or more.

10. The near-infrared oxygen concentration sensor for palpation according to claim 1, wherein
the operation unit calculates a spatial slope of the measurement light based on the measurement light in a plurality of distances between the light emitting unit and the light receiving unit, calculates an absorbance in a tissue of the test subject from the spatial slope, and calculates an oxygenated hemoglobin concentration and an deoxygenated hemoglobin concentration of the test subject from the absorbance.
